SEQUENCE LISTING (16 sequences)

SEQ ID NO: 1 - protein, 8 aa, synthetic construct
GAAGLLGG

SEQ ID NO: 2 - protein, 8 aa, synthetic construct
GPAGLLGG

SEQ ID NO: 3 - protein, 8 aa, synthetic construct
GAAGLRGG

SEQ ID NO: 4 - protein, 8 aa, synthetic construct
GAAGLLGK

SEQ ID NO: 5 - protein, 8 aa, synthetic construct
GAAGLRGK

SEQ ID NO: 6 - protein, 8 aa, synthetic construct
GPAGLRGG

SEQ ID NO: 7 - protein, 8 aa, synthetic construct
GPAGLLGK

SEQ ID NO: 8 - protein, 8 aa, synthetic construct
GPAGLRGK

SEQ ID NO: 9 - protein, 8 aa, synthetic construct
GPPGLTGP

SEQ ID NO: 10 - protein, 8 aa, synthetic construct
  VARIANT 2: note = A or P
  VARIANT 6: note = L or R
  VARIANT 8: note = G or K
GXAGLXGX

SEQ ID NO: 11 - protein, 8 aa, synthetic construct
  VARIANT 6: note = L or R
GPAGLXGK

SEQ ID NO: 12 - protein, 8 aa, synthetic construct
  VARIANT 6: note = L or R
GPAGLXGG

SEQ ID NO: 13 - protein, 8 aa, synthetic construct
  VARIANT 6: note = L or R
GAAGLXGK

SEQ ID NO: 14 - protein, 8 aa, synthetic construct
  VARIANT 6: note = L or R
GAAGLXGG

SEQ ID NO: 15 - protein, 6 aa, synthetic construct
GSGSGS

SEQ ID NO: 16 - protein, 18 aa, synthetic construct
  SITE 5: note = Via its side chain the lysine is coupled to 2,4-dinitrophenyl to form Lys(Dnp)
  SITE 14: note = Via its side chain the lysine is coupled to 7-methoxycoumarin-4-acetic acid to form Lys(Mca)
SGSGKGPAGLLGGKGSGS

(19) United States
(12) Patent Application Publication
(10) Pub. No.: US 2026/0358340 A1
(43) Pub. Date: Nov. 12, 2026

(54) MATRIX METALLOPROTEINASE 13 SENSITIVE PEPTIDES AND SENSORS

(71) Applicant: Julius-Maximilians-Universitaet Wuerzburg, Wuerzburg (DE)

(72) Inventors: Lorenz MEINEL, Wuerzburg (DE); MARC DRIESSEN, Krefeld (DE); Prisca HAMM, Wuerzburg (DE)

(73) Assignee: Julius-Maximilians-Universitaet Wuerzburg, Wuerzburg (DE)

(21) Appl. No.: 19/484,064

(22) PCT Filed: May 17, 2024
(86) PCT No.: PCT/EP2024/063789

§ 371 Date: Nov. 13, 2025

(30) Foreign Application Priority Data
May 17, 2023 (EP) 23174097.8

(51) Int. Cl. G01N 33/573; A61K 47/64; A61K 47/65; C07K 7/06; G01N 33/542; G01N 33/575

(52) U.S. Cl. CPC G01N 33/573; A61K 47/64; A61K 47/65; C07K 7/06; G01N 33/542; G01N 33/5758; G01N 2333/96494; G01N 2410/00; G01N 2800/18

(57) ABSTRACT

The present invention relates to diagnostic MMP-13 sensors, MMP-13 sensitive peptides and related kits. The invention further relates to methods and medical uses involving said sensors, peptides or kits, such as methods for diagnosing cancer or periodontitis in a subject, methods for determining MMP-13 activity as well as the use of the peptide in the treatment of diseases that are related to an increased MMP-13 activity

### PRIORITY

This application corresponds to the U.S. National phase of International Application No. PCT/EP2024/063789, filed May 17, 2024, which, in turn, claims priority to European Patent Application No. 23174097.8 filed May 17, 2023, the contents of which are incorporated by reference herein in their entirety.

### SEQUENCE LISTING

The instant application contains a Sequence Listing that has been submitted in XML format via EFS-Web and is hereby incorporated by reference in its entirety. Said XML copy, created on Nov. 13, 2025, is named LNK_265US_SL.xml and is 18,075 bytes in size.

### FIELD OF THE INVENTION

The present invention relates to the field of protease sensitive peptides, corresponding sensors and their uses in medicine.

### BACKGROUND OF THE INVENTION

Proteases are ubiquitously involved in physiologic and pathologic processes. Protease sensitive linkers (PSL) are peptide sequences that are specifically cleaved by a target enzyme. They can play different roles as part of a bioconjugate [1]. For example, a diagnostic sensor can be developed using a PSL, or an active agent can be released in a disease dependent way.

Matrix metalloproteinase 13 (MMP-13) is a matrix metalloproteinase (MM) that is physiologically significantly involved in the degradation of extracellular matrix. Among others, it gets pathologically upregulated during metastasis of cancer [2], in oral inflammation (such as stomatitis or gingivitis), periodontitis/paradontitis [3] and in degenerative joint diseases [4, 5].

Arthritis is a generic term for more than 100 types of inflammatory joint diseases that are connected with strong pain and severe mobility restraints [6]. Osteoarthritis is the most common form of arthritis and affects more than 500 million people worldwide [7]. It is caused by mechanical abrasion of articular cartilage and, therefore, occurs more often with increasing age. Rheumatoid arthritis is an autoimmune disease, in which the body's own immune system attacks and damages the joints [6].

Prevalence will presumably continue to increase as it did in the last decades [8]. The diseases put a high burden on healthcare due to direct and indirect costs [9].

Frequently, diagnosis occurs only very late [10]. Yet, an early intervention is of relevance in order to prevent irreversible damage. Particularly in early stages, it is important not to impair quality of life by painful intraarticular injections and drug side effects. So far, therapy of arthritis relies on inhibitors of pain and inflammation as well as on immunosuppressant—next to measures not involving medicaments. This administration of disease modifying medicaments is connected with a risk of severe side effects. Once treatment options are exhausted, surgical joint replacement may occur, which is also connected with risks [11, 12].

The key to reaping the benefits of the various treatments is considered the (i) early detection and (ii) broad applicability/accessibility.

In any case, there is a general need in the art to improve diagnosis accordingly.

Specifically, therapy of the above diseases would e.g. benefit greatly if overall diagnosis was shifted to an earlier stage. Consequently, there generally is a need in the art to provide for earlier diagnosis.

Moreover, there is a need in the art for further improvements of diagnosis, such as by addressing a broader population or making testing easier for medical professionals or even at the individual level.

Furthermore, there is a general need in the art for targeted therapy of diseases based on disease markers such as MMP-13.

FRET techniques (where FRET stands for “Forster resonance energy transfer”, which is often also referred to “fluorescence resonance energy transfer” even though it is not restricted to the use of fluorescence) are generally well known to the skilled person—and various variants exist. In principle, the techniques involve a nonradiative transfer of energy between chromophores to e.g. detect interaction of (parts of) molecules. FRET is particularly also useful as a non-destructive method in biological systems such as living cells—for example in the form of FRET biosensors using fluorescent proteins that are compatible with the biological system and which proteins may even be expressed by such systems themselves. Generally, FRET is quite sensitive to the distance between a donor molecule and an acceptor molecule or a donor moiety and an acceptor moiety of one and the same molecule.

A variant of such techniques involves the use of a “FRET pair” including a quencher such as a quenching moiety and a fluorophore. Here, the said quencher/quenching moiety may be located in proximity to said fluorophore and may e.g. be located on the same molecule, which molecule includes a protease sensitive linker (“PSL”). Upon cleavage of the said linker, the fluorophore is easily detectable as the quenching moiety is no longer in close proximity.

### SUMMARY OF THE INVENTION

The present inventors have achieved the above goals by developing MMP-13 sensitive peptides, sensors, and related subject-matter e.g. for periodontitis or cancer (metastasis), which go hand in hand with increased expression of MMP-13 [2, 3]. Particularly also in this context, the concentration of MMP-13 may be quantified via FRET-based PSL.

Moreover, a PSL that is cleavable by MMP-13 as part of a systemically applicable drug conjugate is provided, which comprises an active agent and may additionally comprise a targeting moiety. After having reached its site of action in the affected tissue (such as joints), the active agent can be cleaved off by pathologically upregulated MMP-13 and can achieve its cause-fighting effect. With that, pharmacokinetics is regarded to be optimized and side effects are regarded to be avoided simultaneously.

The present invention provides the subject-matter as defined in the claims.

More specifically, the invention particularly relates to the subject matter defined in the following items [1] to [73]:
[1] A diagnostic sensor comprising,
   a quenching moiety R1,
   a MMP-13-sensitive peptide Pep, and
   a signaling moiety R2,
   wherein the quenching moiety R1 suppresses the signal from the signaling moiety R2 in the said sensor.
[2] The sensor of item [1], wherein
   A) the MMP-13-sensitive peptide Pep comprises amino acid residues having the sequence P4-P3-P2-P1-P1′-P2′-P3′-P4′ (SEQ ID NO: 10), wherein
   P4 is G,
   P3 is independently selected from A and P,
   P2 is A,
   P1 is G,
   P1′ is L,
   P2′ is independently selected from L and R,
   P3′ is G, and
   P4′ is independently selected from G and K,
   or
   B) the MMP-13-sensitive peptide Pep comprises amino acid residues having the sequence GPPGLTGP (SEQ ID NO: 9).
[3] The sensor of any one of items [1] to [2], wherein the MMP-13-sensitive peptide Pep comprises amino acid residues having the sequence P4-P3-P2-P1-P1′-P2′-P3′-P4′ (SEQ ID NO: 11), wherein P4 is G, P3 is P, P2 is A, P1 is G, P1′ is L, P2′ is independently selected from L and R, P3′ is G, and P4′ is K.
[4] The sensor of any one of items [1] to [2], wherein the MMP-13-sensitive peptide Pep comprises amino acid residues having the sequence P4-P3-P2-P1-P1′-P2′-P3′-P4′ (SEQ ID NO: 12), wherein P4 is G, P3 is P, P2 is A, P1 is G, P1′ is L, P2′ is independently selected from L and R, P3′ is G, and P4′ is G.
[5] The sensor of any one of items [1] to [2], wherein the MMP-13-sensitive peptide Pep comprises amino acid residues having the sequence P4-P3-P2-P1-P1′-P2′-P3′-P4′ (SEQ ID NO: 13), wherein P4 is G, P3 is A, P2 is A, P1 is G, P1′ is L, P2′ is independently selected from L and R, P3′ is G, and P4′ is K.
[6] The sensor of any one of items [1] to [2], wherein the MMP-13-sensitive peptide Pep comprises amino acid residues having the sequence P4-P3-P2-P1-P1′-P2′-P3′-P4′ (SEQ ID NO: 14), wherein P4 is G, P3 is A, P2 is A, P1 is G, P1′ is L, P2′ is independently selected from L and R, P3′ is G, and P4′ is G.
[7] The sensor of any one of items [1] to [2], wherein Pep comprises amino acid residues having a sequence selected from the group consisting of GAAGLLGG (SEQ ID NO: 1), GPAGLLGG (SEQ ID NO: 2), GAAGLRGG (SEQ ID NO: 3), GAAGLLGK (SEQ ID NO: 4), GAAGLRGK (SEQ ID NO: 5), GPAGLRGG (SEQ ID NO: 6), GPAGLLGK (SEQ ID NO: 7), GPAGLRGK (SEQ ID NO: 8), and GPPGLTGP (SEQ ID NO: 9).
[8] The sensor of any one of items [1] to [7], wherein Pep comprises amino acid residues having the sequence GAAGLLGG (SEQ ID NO: 1).
[9] The sensor of any one of items [1] to [7], wherein Pep comprises amino acid residues having the sequence GPAGLLGG (SEQ ID NO: 2).
[10] The sensor of any one of items [1] to [9] wherein the sensor comprises the structure R1-Pep-R2.
[11] The sensor of any one of items [1] to [10] wherein the sensor has the structure R1-Pep-R2.
[12] The sensor of any one of items [1] to [11], wherein the quenching moiety R1 comprises a moiety selected from the group consisting of commercially available quenching moieties.
[13] The sensor of any one of items [1] to [12], wherein R1 comprises optionally modified 4-((4-(Dimethylamino)phenyl)azo)benzoic acid, especially wherein R1 is acetylated.
[14] The sensor of any one of items [1] to [13], wherein R1 comprises Ac-K(Dabcyl)-.
[15] The sensor of any one of items [1] to [14], wherein the fluorophore R2 comprises a moiety selected from the group consisting of small organic dyes, fluorescent proteins, and quantum dots.
[16] The sensor of any one of items [1] to [15], wherein R2 comprises 5-((2-Aminoethyl)amino)naphthalene-1-sulfonic acid.
[17] The sensor of any one of items [1] to [16], wherein R2 comprises E(Edans).
[18] The sensor of any one of items [1] to [17], further comprising a first additional moiety L1.
[19] The sensor of anyone of items [1] to [18], further comprising a second additional moiety L2.
[20] The sensor of any one of items [1] to [19], further comprising,
   a first additional moiety L1, and
   a second additional moiety L2.
[21] The sensor of any one of items [18] to [20], wherein the sensor has a structure selected from the group consisting of R1-L1-Pep-L2-R2, L1-R1-Pep-L2-R2, L1-R1-Pep-R2-L2 and R1-L1-Pep-R2-L2.
[22] The sensor of any one of items [18] to [21], wherein L1 comprises a moiety selected from the group consisting of linkers, particularly small peptide linkers, natural polymers and synthetic polymers.
[23] The sensor of any one of items [19] to [22], wherein L2 comprises a moiety selected from the group consisting of linkers, particularly small peptide linkers, natural polymers and synthetic polymers.
[24] The sensor of any one of items [18] to [23], wherein L1 comprises a moiety selected from the group consisting of linkers, particularly small peptide linkers, natural polymers and synthetic polymers, and L2 comprises a moiety selected from the group consisting of linkers, particularly small peptide linkers, natural polymers and synthetic polymers.
[25] The sensor of any one of items [1] to [24], wherein
   i) Pep comprises amino acid residues having a sequence selected from the group consisting of GAAGLLGG (SEQ ID NO: 1), GAAGLRGG (SEQ ID NO: 3), GAAGLLGK (SEQ ID NO: 4), and GAAGLRGK (SEQ ID NO: 5); or
   ii) Pep comprises amino acid residues having a sequence selected from the group consisting of GPAGLLGG (SEQ ID NO: 2), GPAGLRGG (SEQ ID NO: 6), GPAGLLGK (SEQ ID NO: 7), and GPAGLRGK (SEQ ID NO: 8); or
   iii) Pep comprises amino acid residues having the sequence GPPGLTGP (SEQ ID NO: 9).
[26] The sensor of any one of items [1] to [25], wherein Pep comprises amino acid residues having a sequence selected from the group consisting of GAAGLLGG (SEQ ID NO: 1), GAAGLRGG (SEQ ID NO: 3), GAAGLLGK (SEQ ID NO: 4), and GAAGLRGK (SEQ ID NO: 5), particularly wherein said sensor is characterized as a moderate cleaving sensor.
[27] The sensor of any one of items [1] to [25], wherein Pep comprises amino acid residues having a sequence selected from the group consisting of GPAGLLGG (SEQ ID NO: 2), GPAGLRGG (SEQ ID NO: 6), GPAGLLGK (SEQ ID NO: 7), and GPAGLRGK (SEQ ID NO: 8), particularly wherein said sensor is characterized as a fast cleaving sensor.
[28] The sensor of any one of items [1] to [25], wherein Pep comprises amino acid residues having the sequence GPPGLTGP (SEQ ID NO: 9), particularly wherein said sensor is characterized as a slow cleaving sensor.
[29] The sensor of any one of items [1] to [25], wherein Pep comprises amino acid residues having a sequence selected from the group consisting of GPAGLLGG (SEQ ID NO: 2), GAAGLRGG (SEQ ID NO: 3), and GAAGLLGK (SEQ ID NO: 4).
[30] The sensor of any one of items [1] to [25], wherein Pep comprises amino acid residues having a sequence selected from the group consisting of GAAGLRGK (SEQ ID NO: 5), GPAGLRGG (SEQ ID NO: 6), GPAGLLGK (SEQ ID NO: 7), and GPAGLRGK (SEQ ID NO: 8).
[31] The sensor of any one of items [1] to [30] for use in medicine.
[32] The sensor of any one of items [1] to [31] for use in a method of diagnosing cancer, particularly wherein an increased level of MMP-13 activity relative to a control level is indicative of the subject having cancer.
[33] The sensor for use of item [32], wherein the method comprises:
a) determining a level of MMP-13 activity in the subject,
b) optionally comparing the level of MMP-13 activity to a control level of MMP-13 activity,
c) diagnosing whether the subject has cancer, wherein an increased level of MMP-13 activity is indicative of the subject having cancer.
[34] The sensor of any one of items [1] to [30] for use in a method of diagnosing cancer metastasis, particularly wherein the method is further defined as in any of items [32] to [33].
[35] The sensor of any one of items [1] to [30] for use in a method of diagnosing periodontitis, particularly wherein an increased level of MMP-13 activity relative to a control level is indicative of the subject having periodontitis.
[36] The sensor for use of item [35], wherein the method comprises:
a) determining a level of MMP-13 activity in the subject,
b) optionally comparing the level of MMP-13 activity to a control level of MMP-13 activity,
c) diagnosing whether the subject has cancer, wherein an increased level of MMP-13 activity is indicative of the subject having periodontitis.
[37] The sensor for use of any one of items [32] to [36], wherein the subject is a mammalian subject, particularly a human subject.
[38] The sensor for use of any one of items [32] to [37], wherein the method comprises determining MMP-13 activity in the oral cavity of the subject,
[39] A method of determining MMP-13 activity in a sample, wherein the method comprises using a sensor in accordance with any one of items [1] to [30].
[40] The method of item [39] wherein the sample has been obtained from a subject, particularly a mammalian subject, especially a human subject.
[41] The method of any one of items [39] to [40], wherein the method is a method of diagnosing cancer.
   particularly wherein an increased level of MMP-13 activity relative to the level of a control is indicative of the subject having cancer.
[42] The method of item [41], wherein the method comprises
a) determining a level of MMP-13 activity in a sample obtained from the subject,
b) optionally comparing the level in the said sample to a control level of MMP-13 activity,
c) diagnosing whether the subject has cancer, wherein an increased level of MMP-13 activity is indicative of the subject having cancer.
[43] The method of any one of items [39] to [40], wherein the method is a method of diagnosing cancer metastasis.
[44] The method of item [43], wherein the method is further defined as in any of items [41] to [42].
[45] The method of any one of items [39] to [40], wherein the method is a method of diagnosing periodontitis,
   particularly wherein an increased level of MMP-13 activity relative to the level of a control is indicative of the subject having periodontitis.
[46] The method of item [45], wherein the method comprises
a) determining a level of MMP-13 activity in a sample obtained from the subject,
b) optionally comparing the level in the said sample to a control level of MMP-13 activity,
c) diagnosing whether the subject has periodontitis, wherein an increased level of MMP-13 activity is indicative of the subject having periodontitis.
[47] The method of any one of items [39] to [46], wherein the sample has been obtained from the oral cavity of a subject.
[48] A MMP-13-sensitive peptide having a length of from 8 to 16 amino acids, wherein
   A) the MMP-13-sensitive peptide comprises amino acid residues having the sequence P4-P3-P2-P1-P1′-P2′-P3′-P4′ (SEQ ID NO: 10), wherein
   P4 is G,
   P3 is independently selected from A and P,
   P2 is A,
   P1 is G,
   P1′ is L,
   P2′ is independently selected from L and R,
   P3′ is G, and
   P4′ is independently selected from G and K,
   or
   B) the MMP-13-sensitive peptide comprises amino acid residues having the sequence GPPGLTGP (SEQ ID NO: 9).
[49] The peptide of item [48] comprising amino acid residues having the sequence P4-P3-P2-P1-P1′-P2′-P3′-P4′ (SEQ ID NO: 10), wherein
   P4 is G,
   P3 is independently selected from A and P,
   P2 is A,
   P1 is G,
   P1′ is L,
   P2′ is independently selected from L and R,
   P3′ is G, and
   P4′ is independently selected from G and K.
[50] The peptide of any one of items [48] to [49] comprising amino acid residues having the sequence P4-P3-P2-P1-P1′-P2′-P3′-P4′ (SEQ ID NO: 11), wherein P4 is G, P3 is P, P2 is A, P1 is G, P1′ is L, P2′ is independently selected from L and R, P3′ is G, and P4′ is K.
[51] The peptide of any one of items [48] to [49] comprising amino acid residues having the sequence P4-P3-P2-P1-P1′-P2′-P3′-P4′ (SEQ ID NO: 12), wherein P4 is G, P3 is P, P2 is A, P1 is G, P1′ is L, P2′ is independently selected from L and R, P3′ is G, and P4′ is G.
[52] The peptide of any one of items [48] to [49] comprising amino acid residues having the sequence P4-P3-P2-P1-P1′-P2′-P3′-P4′ (SEQ ID NO: 13), wherein P4 is G, P3 is A, P2 is A, P1 is G, P1′ is L, P2′ is independently selected from L and R, P3′ is G, and P4′ is K.
[53] The peptide of any one of items [48] to [49] comprising amino acid residues having the sequence P4-P3-P2-P1-P1′-P2′-P3′-P4′ (SEQ ID NO: 14), wherein P4 is G, P3 is A, P2 is A, P1 is G, P1′ is L, P2′ is independently selected from L and R, P3′ is G, and P4′ is G.
[54] The peptide of any one of items [48] to [49] comprising amino acid residues having a sequence selected from the group consisting of GAAGLLGG (SEQ ID NO: 1), GPAGLLGG (SEQ ID NO: 2), GAAGLRGG (SEQ ID NO: 3), GAAGLLGK (SEQ ID NO: 4), GAAGLRGK (SEQ ID NO: 5), GPAGLRGG (SEQ ID NO: 6), GPAGLLGK (SEQ ID NO: 7), GPAGLRGK (SEQ ID NO: 8), and GPPGLTGP (SEQ ID NO: 9).
[55] The peptide of any one of items [48] to [49] comprising amino acid residues having the sequence GAAGLLGG (SEQ ID NO: 1).
[56] The peptide of any one of items [48] to [49] comprising amino acid residues having the sequence GPAGLLGG (SEQ ID NO: 2).
[57] The peptide of any one of items [48] or [54] comprising amino acid residues having the sequence GPPGLTGP (SEQ ID NO: 9),
   particularly wherein said peptide is characterized as a slow cleaving peptide.
[58] The peptide of any one of items [48] to [54] comprising amino acid residues having a sequence selected from the group consisting of GAAGLLGG (SEQ ID NO: 1), GAAGLRGG (SEQ ID NO: 3), GAAGLLGK (SEQ ID NO: 4), and GAAGLRGK (SEQ ID NO: 5), particularly wherein said peptide is characterized as a moderate cleaving peptide.
[59] The peptide of any one of items [48] to [54] comprising amino acid residues having a sequence selected from the group consisting of GPAGLLGG (SEQ ID NO: 2), GPAGLRGG (SEQ ID NO: 6), GPAGLLGK (SEQ ID NO: 7), and GPAGLRGK (SEQ ID NO: 8), particularly wherein said peptide is characterized as a fast cleaving peptide.
[60] The peptide of any one of items [48] to [54] comprising amino acid residues having a sequence selected from the group consisting of GPAGLLGG (SEQ ID NO: 2), GAAGLRGG (SEQ ID NO: 3), and GAAGLLGK (SEQ ID NO: 4),
[61] The peptide of any one of items [48] to [54] comprising amino acid residues having a sequence selected from the group consisting of GAAGLRGK (SEQ ID NO: 5), GPAGLRGG (SEQ ID NO: 6), GPAGLLGK (SEQ ID NO: 7), and GPAGLRGK (SEQ ID NO: 8).
[62] The peptide of any one of items [48] to [61] for use in medicine.
[63] The peptide of any one of items [48] to [62] for use in a diagnostic method, particularly wherein the method is further defined in accordance with any one of items [32] to [38].
[64] A method of determining MMP-13 activity in a sample, wherein the method comprises using a peptide in accordance with any one of items [48] to [61],
   particularly wherein the method is further defined in accordance with any one of items [39] to [47].
[65] A diagnostic kit comprising a sensor in accordance with any one of items [1] to [30].
[66] A diagnostic kit comprising a peptide in accordance with any one of items [48] to [61].
[67] The kit in accordance with any one of items [65] to [66] for use in medicine, particularly for use in a diagnostic method as further defined as in any one of items [32] to [38].
[68] A method of determining MMP-13 activity in a sample, wherein the method comprises using a kit in accordance with any one of items [65] to [66], particularly wherein the method is further defined in accordance with any one of items [39] to [47].
[69] The peptide of any one of items [48] to [61] for use in a method of preventing or treating a disease.
[70] The peptide for use of item [69], wherein the disease selected from the group consisting of cancer, oral inflammation, periodontitis, and arthropathies,
   particularly wherein the arthropathy is selected from degenerative arthropathies (such as osteoarthritis) and inflammatory arthropathies (such as rheumatoid arthritis), and wherein oral inflammation is selected from stomatitis and gingivitis.
[71] The peptide for use of any one of items [69] to [70], wherein the peptide is part of a drug conjugate, comprising the peptide as well as an active agent, wherein the active agent is released from the drug conjugate upon cleavage with MMP-13.
[72] The peptide for use of any one of items [69] to [71], wherein the active agent is selected from small molecule drugs,
   preferably selected from the group consisting of
   1) nonsteroidal anti-inflammatory drugs (particularly selected from non-selective COX-1/2 inhibitors such as oxicame (piroxicame), arylpropionic acid derivatives (e.g. ibuprofen) or arylacetic acid derivatives (e.g. diclofenac) or from selective COX-2 inhibitors such as celecoxib or etoricoxib,
   2) corticosteroids such as cortisol,
   3) synthetic disease-modifying antirheumatic drug (sDMARD) (preferably selected from conventional sDMARD (csDMARD) such as methotrexate or sulfasalazine, or from targeting sDMARD (tsDMARD) such as ruxolitinib), and
   4) antiinfectives, such as antibiotics, in particular tetracycline antibiotics (such as doxycycline or minocycline).
[73] The peptide for use of any one of items [69] to [71], wherein the active agent is selected from biologic drugs,
   preferably selected from the group consisting of
   1) monoclonal antibodies (such as adalimumab)
   and
   2) biologic antagonists/inhibitors (such as anakinra etanercept).
[74] The peptide for use of any one of items [69] to [73], wherein drug conjugate further comprises a targeting moiety,
   preferably wherein the targeting moiety is selected from the group consisting of
   1) small molecules (such as folate),
   and
   2) biologics, preferably selected from the group consisting of monoclonal antibodies (such as Her-2 antibodies), affibodies, darpins and targeting peptides.

### BRIEF DESCRIPTION OF THE FIGURES

FIG. 1: Schematic view of the work flow. (A) Selection of the target protease. (B) Identification of protease sites. (C) Synthesis of the resulting peptides and testing with the target protease.

FIG. 2: Proteomic Identification of protease Cleavage Sites (PICS) Heat maps for MMP-13. (A) E. coli Trypsin Library. (B) E. coli GluC Library.

FIG. 3: ESI-TOF Charakterisierung der beschriebenen Peptide. (A) Calculated m/z: 1232.6, measured m/z: 1232.5, (B) Calculated m/z: 1258.6, measured m/z: 1258.5, (C) Calculated m/z: 1275.6, measured m/z: 1275.6, (D) Calculated m/z: 1303.6, measured m/z: 1304.7, (E) Calculated m/z: 1346.7, measured m/z: 1346.8, (F) Calculated m/z: 1301.6, measured m/z: 1301.5, (G) Calculated m/z: 1329.7, measured m/z: 1329.6, (H) Calculated m/z: 1372.7, measured m/z: 1372.7, (1) Calculated m/z: 1312.6, measured m/z: 1312.6.

FIG. 4: High Performance Liquid Chromatography (HPLC) Purity analyses of the disclosed peptides.

FIG. 5: MMP cleavage behavior of the disclosed peptides. (A) MMP-13 Cleavage behavior of the disclosed peptides over a time range of 24 h. (B) Fraction of cleaved peptides after incubation with .MMP-13, MMP-8 and MMP-9 after 24 h.

FIG. 6: Plasma stability of the peptides after 24 h.

FIG. 7: Inhibition of MMP13, MMP8 and MMP9 by EDTA, Marimastat, SDS and a commercial protease inhibitor mixture (see Example 4).

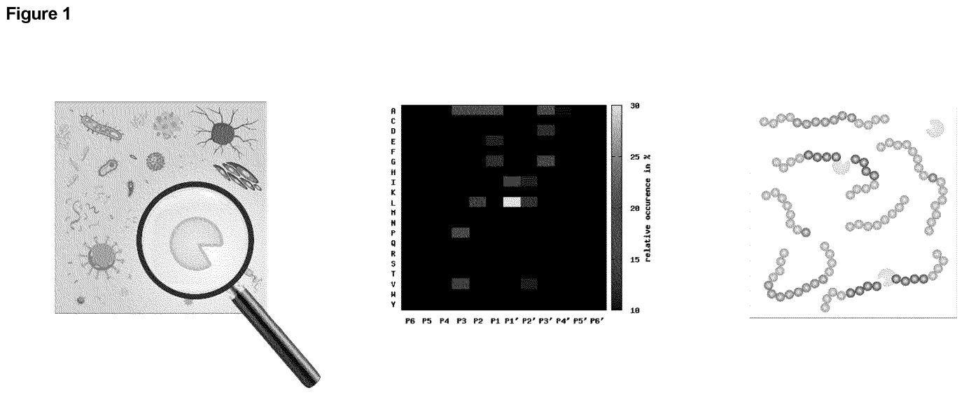

### DETAILED DESCRIPTION OF THE PREFERRED EMBODIMENTS

One object of the present invention was the development of MMP-13 sensitive peptides, e.g. for their use in diagnostic and therapeutic systems relating to diseases that are connected to (pathologically) increased MMP concentration.

Among the peptides/sensors developed in accordance with the invention, there are peptides having different features, as they are e.g.
cleavable by MMP-13, MMP-8 and MMP-9
cleavable by MMP-13, but only to a minimal extent by MMP-8 and MMP-9

Moreover, next to varying specificity, the peptides of the invention are cleaved by MMP-13 with different kinetics. Overall, a respective selection offers various possible uses.

Due to an increased MMP-13 activity in various diseases, the present inventors consider this protease as a promising biomarker or a promising trigger to release an active agent.

For example, in accordance with the invention, peptides cleavable by MMP-13 are regarded to enable an (early) diagnosis of diseases involving upregulation of MMP-13.

In addition, in accordance with the invention, an active agent may be released from a bioconjugate, more specifically a drug conjugate. Consequently, in more detail, said active agent may exert its effect in response to pathologic MMP-13 activity. Release kinetics may be varied in dependent of the cleavage rate of the respective peptide (linker).

In addition, the subject-matter of the invention is considered to specifically e.g. involve the following advantages:
1.) Provision of novel diagnostic MMP-13 sensors.
2.) Provision of rapid diagnostic tools.
3.) Provision of versatile, adaptable diagnostic tools (e.g. PSL exchange)
4.) Provision of easy-to-use diagnostic tools (e.g. for health care professionals)
5.) Provision of therapeutic tools, particularly for specific and targeted therapy.

Specifically, the present invention provides the subject-matter as defined in the claims.

In particular, the present invention provides diagnostic MMP-13 sensors, respective MMP-13 sensitive peptides and related kits, methods and medical uses involving said sensors, peptides or kits, such as diagnostic methods, as well as methods for determining MMP-13 activity.

In a first aspect, the present invention relates a diagnostic sensor comprising a quenching moiety R1, a MMP-13-sensitive peptide Pep, and a signaling moiety R2.

Without intending to be bound by theory, the said quenching moiety R1 suppresses the signal from the signaling moiety R2 in the said sensor. Consequently, once the MMP-13-sensitive peptide has been cleaved by MMP-13, the signaling moiety is easily detectable due to spatial absence of the quenching moiety.

In a set of preferred embodiments of the first aspect, the MMP-13-sensitive peptide Pep comprises amino acid residues having the sequence P4-P3-P2-P1-P1′-P2′-P3′-P4′, wherein P4 is G, P3 is independently selected from A and P, P2 is A, P1 is G, P1′ is L, P2′ is independently selected from L and R, P3′ is G, and P4′ is independently selected from G and K.

Alternatively, in the first aspect, the MMP-13-sensitive peptide Pep comprises amino acid residues having the sequence GPPGLTGP (SEQ ID NO: 9).

In preferred embodiments, said MMP-13-sensitive peptide Pep comprises amino acid amino residues having a sequence selected from the group consisting of GAAGLLGG (SEQ ID NO: 1), GPAGLLGG (SEQ ID NO: 2), GAAGLRGG (SEQ ID NO: 3), GAAGLLGK (SEQ ID NO: 4), GAAGLRGK (SEQ ID NO: 5), GPAGLRGG (SEQ ID NO: 6), GPAGLLGK (SEQ ID NO: 7), GPAGLRGK (SEQ ID NO: 8), and GPPGLTGP (SEQ ID NO: 9).

In a further set of preferred embodiments of the first aspect, the MMP-13-sensitive peptide Pep comprises amino acid residues selected from the following group:
i) amino acid residues having the sequence P4-P3-P2-P1-P1′-P2′-P3′-P4′ (SEQ ID NO: 11), wherein P4 is G, P3 is P, P2 is A, P1 is G, P1′ is L, P2′ is independently selected from L and R, P3′ is G, and P4′ is K,
ii) amino acid residues having the sequence P4-P3-P2-P1-P1′-P2′-P3′-P4′ (SEQ ID NO: 12), wherein P4 is G, P3 is P, P2 is A, P1 is G, P1′ is L, P2′ is independently selected from L and R, P3′ is G, and P4′ is G,
iii) amino acid residues having the sequence P4-P3-P2-P1-P1′-P2′-P3′-P4′ (SEQ ID NO: 13), wherein P4 is G, P3 is A, P2 is A, P1 is G, P1′ is L, P2′ is independently selected from L and R, P3′ is G, and P4′ is K,
iv) amino acid residues having the sequence P4-P3-P2-P1-P1′-P2′-P3′-P4′ (SEQ ID NO: 14), wherein P4 is G, P3 is A, P2 is A, P1 is G, P1′ is L, P2′ is independently selected from L and R, P3′ is G, and P4′ is G.

In a particular embodiment, the MMP-13-sensitive peptide Pep comprises amino acid residues having the sequence GAAGLLGG (SEQ ID NO: 1).

In a particular embodiment, the MMP-13-sensitive peptide Pep comprises amino acid residues having the sequence GPAGLLGG (SEQ ID NO: 2).

In a further set of preferred embodiments of the first aspect, the MMP-13-sensitive peptide Pep comprises amino acid residues selected from the following group: GAAGLLGG (SEQ ID NO: 1), GAAGLRGG (SEQ ID NO: 3), GAAGLLGK (SEQ ID NO: 4), and GAAGLRGK (SEQ ID NO: 5), particularly wherein said peptide/sensor is characterized as a moderate cleaving sensor.

In a further set of preferred embodiments of the first aspect, the MMP-13-sensitive peptide Pep comprises amino acid residues selected from the following group: GPAGLLGG (SEQ ID NO: 2), GPAGLRGG (SEQ ID NO: 6), GPAGLLGK (SEQ ID NO: 7), and GPAGLRGK (SEQ ID NO: 8), particularly wherein said peptide/sensor is characterized as a fast cleaving sensor.

Furthermore, the sensor may comprise the structure R1-Pep-R2. Alternatively, the sensor may have the structure R1-Pep-R2.

Generally in context with the present invention, particular additional embodiments for all aspects and embodiments described herein correspond to embodiments, where the term(s) “comprises” (and suchlike terms) are replaced by the term “has” (and suchlike terms).

Generally in context with the present invention, particular even further embodiments for all aspects and embodiments described herein correspond to embodiments, where the term(s) “comprises” (and suchlike terms) are replaced by the term “consists of” (and suchlike terms).

In specific embodiments of the first aspect, the quenching moiety R1 comprises a moiety selected from the group consisting of commercially available quenching moieties, particularly selected from the group consisting of Dabcyl and the quenching moieties commercially available under the trade names Dark Fluorescent Quencher, Black Hole Quenchers, Tide Quencher, BlackBerry Quencher, Qxl quencers, Iowa Black FW, Iowa Black RQ, IRDye QC-1, Eclipse.

In any case, such quenchers are well known to and readily available by the person skilled in the art and the above list is a non-limiting list of particular examples. In specific embodiments, R1 comprises optionally modified 4-((4-(Dimethylamino)phenyl)azo)benzoic acid (also known as “Dabcyl”, especially wherein R1 is acetylated. In a further particular embodiment, R1 comprises Ac-K(Dabcyl).

In particular embodiments of the first aspect, the fluorophore R2 comprises a moiety selected from the group consisting of small organic dyes, fluorescent proteins, and quantum dots.

In particular embodiments, the fluorophore R2 comprises a small organic dye, particularly selected from the group consisting of fluoresceins, rhodamines and particular commercially available small organic dyes (particularly selected from CF-Dyes, Alexa Fluor, DyLight Fluor, and Atto).

In particular embodiments, the fluorophore R2 comprises a fluorescent protein, particularly selected from GFP, CFP and particular commercially available fluorophores (such as mCherry).

In particular embodiments, the fluorophore R2 comprises a quantum dot.

Generally, suitable fluorophores are not particularly limited and will readily be selected and are readily obtainable by the skilled person.

In specific embodiments, R2 comprises 5-((2-Aminoethyl)amino)naphthalene-1-sulfonic acid (also known as “Edans”). In a further particular embodiment, R2 comprises E(Edans).

In a further set of embodiments of the first aspect, the sensor further comprises first additional moiety L1, and/or a second additional moiety L2.

in particular embodiments, the sensor comprises a structure selected from the group consisting of R1-L1-Pep-L2-R2, L1-R1-Pep-L2-R2, L1-R1-Pep-R2-L2 and R1-L2-Pep-R2-L2.

In more specific embodiments, the sensor has a structure selected from the group consisting of R1-L1-Pep-L2-R2, L1-R1-Pep-L2-R2, L1-R1-Pep-R2-L2 and R1-L1-Pep-R2-L2.

Preferably, L1 comprises a moiety selected from the group consisting of linkers, particularly small peptide linkers, natural polymers and synthetic polymers.

Preferably, L2 comprises a moiety selected from the group consisting of linkers, particularly small peptide linkers, natural polymers and synthetic polymers.

In particular embodiments, the linker is a small peptide linker. In particular embodiments, the linker is GSGSGS (SEQ ID NO: 15). This is only one non-limiting example, as the skilled person will readily be in a position to select and use suitable linkers for L1 and/or L2.

In particular embodiments, the natural polymer is cellulose. This is only one non-limiting example, as the skilled person will readily be in a position to select and use suitable polymers for L1 and/or L2.

In particular embodiments, the synthetic polymer is selected from the group consisting of polyoxazoline, polyethylene glycols, polycaprolactone, and poly(vinyl)alcohols. These are only some non-limiting examples, as the skilled person will readily be in a position to select and use suitable polymers for L1 and/or L2.

Moreover, the skilled person will readily be in a position to select preferred additional moieties L1 and L2 as well as preferred pairs for L1 and L2 depending on the desired application. Such selections may e.g. be taken in view of a desired increase of hydrophilic properties, desired chemical properties, desired introduction of further functionalities and suchlike.

In a second aspect, the present invention relates to the sensor of the first aspect for use in medicine.

In a third aspect, the present invention relates to the sensor of the first aspect for use in a diagnostic method, and particularly for use in a method of diagnosing a condition.

Preferably, said condition is selected from cancer, cancer metastasis, and periodontitis.

In certain embodiments of the latter aspects, the use or method, respectively, comprises determining MMP-13 activity in a subject, particularly in the oral cavity of the subject.

In preferred embodiments of the second and third aspects, an increased level of MMP-13 activity relative to a control level is indicative of the subject having said condition.

In particular embodiments herein, an increased level of MMP-13 activity is indicated by a fold change of at least 5×, particularly in a time of 5-20 minutes, more preferably between 5-10 minutes. Especially, said increased level is determined relative to a control level established using a control without said condition.

Depending on the particular embodiment/aspect herein, increased levels of MMP-13 activity may be determined in a subject, particularly in the oral cavity of the subject, or in a sample obtained from the subject.

In particular embodiments of the third aspect, the method is a method of diagnosing a subject for a said condition, and comprises:
a) determining a level of MMP-13 activity in the subject,
b) diagnosing whether the subject has the condition, wherein an increased level of MMP-13 activity is indicative of the subject having the condition.

In more specific embodiments of the third aspect, the method comprises
a) determining a level of MMP-13 activity in the subject,
b) comparing the level of MMP-13 activity to a control level of MMP-13 activity, and
c) diagnosing whether the subject has the condition, wherein an increased level of MMP-13 activity is indicative of the subject having the condition.

In accordance with the present invention, the subject preferably is a mammalian subject, particularly a human subject.

Generally, other preferred embodiments of the third aspect correspond to those of the first aspect.

In a fourth aspect, the present invention relates to a method of determining MMP-13 activity in a sample, wherein the method comprises using a sensor in accordance with the first aspect.

In preferred embodiments, the sample has been obtained from a subject, particularly a mammalian subject, especially a human subject.

In particular embodiments, the sample has been obtained from the oral cavity of a subject. In specific embodiments, the sample includes cells from the subject, particularly mucosal tissue. In alternative specific embodiments, the sample includes components of cells from the subject, such as components of mucosal tissue obtained via a mucosal swap.

In a set of preferred embodiments of the fourth aspect, the method is a method of diagnosing a condition, in particular wherein an increased level of MMP-13 activity relative to the level of a control is indicative of the subject having said condition.

Preferably, said condition is selected from cancer, cancer metastasis, and periodontitis

In particular embodiments of the fourth aspect, the method comprises
a) determining a level of MMP-13 activity in a sample obtained from the subject,
b) optionally comparing the level in the said sample to a control level of MMP-13 activity,
c) diagnosing whether the subject has the condition, wherein an increased level of MMP-13 activity is indicative of the subject having the condition.

Additional preferred embodiments of the fourth aspect correspond to those of the second and third aspects.

In a fifth aspect, the present invention relates to a MMP-13-sensitive peptide having a length of from 8 to 16 amino acids, wherein the MMP-13-sensitive peptide comprises amino acid residues having the sequence P4-P3-P2-P1-P1′-P2′-P3′-P4′ (SEQ ID NO: 10), wherein P4 is G, P3 is independently selected from A and P, P2 is A, P1 is G, P1′ is L, P2′ is independently selected from L and R, P3′ is G, and P4′ is independently selected from G and K.

Alternatively, the fifth aspect relates to a MMP-13-sensitive peptide having a length of from 8 to 16 amino acids and comprising amino acid residues having the sequence GPPGLTGP (SEQ ID NO: 9).

In preferred embodiments of the fifth aspect, said peptide comprises amino acid amino residues having a sequence selected from the group consisting of GAAGLLGG (SEQ ID NO: 1), GPAGLLGG (SEQ ID NO: 2), GAAGLRGG (SEQ ID NO: 3), GAAGLLGK (SEQ ID NO: 4), GAAGLRGK (SEQ ID NO: 5), GPAGLRGG (SEQ ID NO: 6), GPAGLLGK (SEQ ID NO: 7), GPAGLRGK (SEQ ID NO: 8), and GPPGLTGP (SEQ ID NO: 9).

In a further set of preferred embodiments of the fifth aspect, the peptide comprises amino acid residues selected from the following group:
i) amino acid residues having the sequence P4-P3-P2-P1-P1′-P2′-P3′-P4′ (SEQ ID NO: 11), wherein P4 is G, P3 is P, P2 is A, P1 is G, P1′ is L, P2′ is independently selected from L and R, P3′ is G, and P4′ is K,
ii) amino acid residues having the sequence P4-P3-P2-P1-P1′-P2′-P3′-P4′ (SEQ ID NO: 12), wherein P4 is G, P3 is P, P2 is A, P1 is G, P1′ is L, P2′ is independently selected from L and R, P3′ is G, and P4′ is G,
iii) amino acid residues having the sequence P4-P3-P2-P1-P1′-P2′-P3′-P4′ (SEQ ID NO: 13), wherein P4 is G, P3 is A, P2 is A, P1 is G, P1′ is L, P2′ is independently selected from L and R, P3′ is G, and P4′ is K,
iv) amino acid residues having the sequence P4-P3-P2-P1-P1′-P2′-P3′-P4′ (SEQ ID NO: 14), wherein P4 is G, P3 is A, P2 is A, P1 is G, P1′ is L, P2′ is independently selected from L and R, P3′ is G, and P4′ is G.

In a particular embodiment, the peptide comprises amino acid residues having the sequence GAAGLLGG (SEQ ID NO: 1).

In a particular embodiment, the peptide comprises amino acid residues having the sequence GPAGLLGG (SEQ ID NO: 2).

In a further set of preferred embodiments of the first aspect, the peptide comprises amino acid residues selected from the following group: GAAGLLGG (SEQ ID NO: 1), GAAGLRGG (SEQ ID NO: 3), GAAGLLGK (SEQ ID NO: 4), and GAAGLRGK (SEQ ID NO: 5), particularly wherein said peptide is characterized as a moderate cleaving peptide.

In a further set of preferred embodiments of the first aspect, peptide comprises amino acid residues selected from the following group: GPAGLLGG (SEQ ID NO: 2), GPAGLRGG (SEQ ID NO: 6), GPAGLLGK (SEQ ID NO: 7), and GPAGLRGK (SEQ ID NO: 8), particularly wherein said peptide is characterized as a fast cleaving peptide.

In further embodiments of the fifth aspect, the peptide further comprises amino acids selected from glycine and serine. In a more specific embodiment, the peptide comprises one of the sequences of eight amino acid residues as defined herein, whereas the other eight amino acid residues are selected from glycine and serine. More particularly, each end of the eight amino acid residues as defined herein comprises two repetitions of glycine and serine.

In even further embodiments of the fifth aspect, the peptide is further characterized in line with further embodiments of the first aspect.

In a sixth aspect, the present invention relates to the peptides of the fifth aspect for use in medicine. Consequently, preferred embodiments of the sixth aspect correspond to those of the first and second aspects.

In a seventh aspect, the present invention relates to the peptides of the fifth aspect for use in a diagnostic method of the third aspect. Consequently, preferred embodiments of the seventh aspect correspond to those of the first and third aspects.

In an eighth aspect, the present invention relates to a method of determining MMP-13 activity in a sample, wherein the method comprises using a peptide in accordance with the fifth aspect. Consequently, preferred embodiments of the eighth aspect correspond to those of the first and fourth aspects.

In a ninth aspect, the present invention relates to a kit comprising the sensor of the first aspect. Consequently, preferred embodiments of the ninth aspect correspond to those of the first aspect.

In a tenth aspect, the present invention relates to a kit comprising the peptide of the fifth aspect. Consequently, preferred embodiments of the tenth aspect correspond to those of the first and fifth aspect.

In an eleventh aspect, the present invention relates the kits of the ninth and tenth aspects for use in medicine, particularly for use in diagnostic methods. Consequently, preferred embodiments of the eleventh aspect correspond to those of the first, third, and seventh aspects.

In a twelfth aspect, the present invention relates to a method of determining MMP-13 activity in a sample, wherein the method comprises using a kit in accordance with the ninth or tenth aspect. Consequently, preferred embodiments of the twelfth aspect correspond to those of the first and fourth aspects.

In a thirteenth aspect, the present invention relates to the peptide of the fifth aspect for use in a method of preventing or treating a disease.

In preferred embodiments, the disease is selected from the group consisting of cancer, oral inflammation, periodontitis/paradontitis, and arthropathies.

Preferably, oral inflammation is selected from stomatitis and gingivitis.

In particular, the arthropathy is selected from degenerative arthropathies and inflammatory arthropathies.

Preferably, the degenerative arthropathy is osteoarthritis

Preferably, the inflammatory arthropathy is rheumatoid arthritis.

Further preferred embodiments of the thirteenth aspect correspond to those described in the context with the first and fifth aspects.

In context with the thirteenth aspect, the peptide is part of a drug conjugate, comprising the peptide as well as an active agent. Preferably, the active agent is released from the drug conjugate upon cleavage with MMP-13.

In certain embodiment, the active agent is selected from small molecule drugs, preferably selected from the group consisting of
1) nonsteroidal anti-inflammatory drugs (particularly selected from non-selective COX-1/2 inhibitors such as oxicame (piroxicame), arylpropionic acid derivatives (e.g. ibuprofen) or arylacetic acid derivatives (e.g. diclofenac) or from selective COX-2 inhibitors such as celecoxib or etoricoxib,
2) corticosteroids such as cortisol,
3) synthetic disease-modifying antirheumatic drug (sDMARD) (preferably selected from conventional sDMARD (csDMARD) such as methotrexate or sulfasalazine, or from targeting sDMARD (tsDMARD) such as ruxolitinib), and
4) antiinfectives, such as antibiotics, in particular tetracycline antibiotics (such as doxycycline or minocycline).

In further embodiments, the active agent is selected from biologic drugs, preferably selected from the group consisting of 1) monoclonal antibodies (such as adalimumab) and 2) biologic antagonists/inhibitors (such as anakinra etanercept). As the skilled person will readily appreciate, the above small molecule examples 1), 2) and 3) are particularly suited in context with arthropathies (joint disease), whereas the above small molecule examples 4) are particularly suited in context with periodontitis, and whereas also in cancer therapy, the active agent may be selected from small molecules or from biologics (such as monoclonal antibodies, antagonists/inhibitors).

As the skilled person will readily appreciate, e.g. in case of joint diseases, the therapy may also occur stepwise, such as using the above examples 1)-2)-3), whereas biologic drugs may be used as an alternative for “3)”.

In further embodiments in context with the thirteenth aspect, the said drug conjugate further comprises a targeting moiety. Preferably, the targeting moiety is selected from the group consisting of 1) small molecules (such as folate), and 2) biologics, preferably selected from the group consisting of monoclonal antibodies (such as Her-2 antibodies), affibodies, darpins and targeting peptides.

The studies of the present inventors underlying the present invention will be described in further detail in the example section.

### EXAMPLES

Next, the invention will further be described by reference to the following examples. Importantly, any examples herein are intended for further illustrating the invention, but are by no means intended to limit the invention in any way.

### Example 1

The method of Proteomic Identification of Protease Cleavage Sites (PICS) was utilized for examining the cleavage specificity of MMP-13.

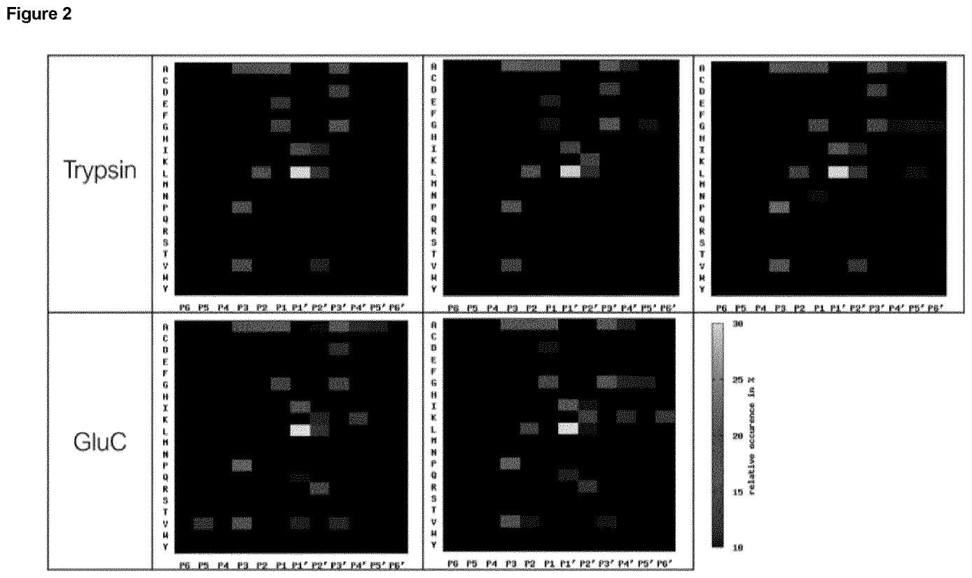

Using PICS (cf. FIG. 2) a preference of MMP-13 for the following amino acids at the cleavage site was identified:

P4: alanine
P3: alanine or proline
P2: alanine
P1: alanine
P1′: leucine
P2′: leucine or arginine
P3′: alanine
P4′: alanine or lysine

As the increased occurrence of hydrophobic amino acids may involve problems during synthesis and as regards solubility, the present inventors eventually replaced alanine at P4, P1, P3 and P4′ by glycine. At these positions (and as opposed to P3 and P2), preference for alanine as opposed to glycine turned out to only be slightly bigger.

At each of positions P3, P2′ and P4′, two amino acids occurred more frequently. Each possible combination was tested, which resulted in a total of eight sequences.

The present inventors concluded from the results of the PICS assays that the specificity of MMP-13 does not extend beyond positions P4 to P4′.

For that reason, two repetitions each of glycine and serine were added before and after each sequence. These amino acids were considered to further improve solubility while not having large side chains that might interfere with the cleavage by MMP-13.

In summary, based on all the obtained results, eight specific peptide sequences were derived, optimized and synthesized.

Another peptide sequence was established based on literature search involving an MMP-13-substrate (alpha-1-type-II-collagen), which is mainly composed by amino acids occurring most frequently at the respective positions of the cleavage site.

### Example 2

In the following, the resulting peptides were tested with different MMPs and were analyzed as to their cleavage rates, which yielded the respective specific features of the peptides.

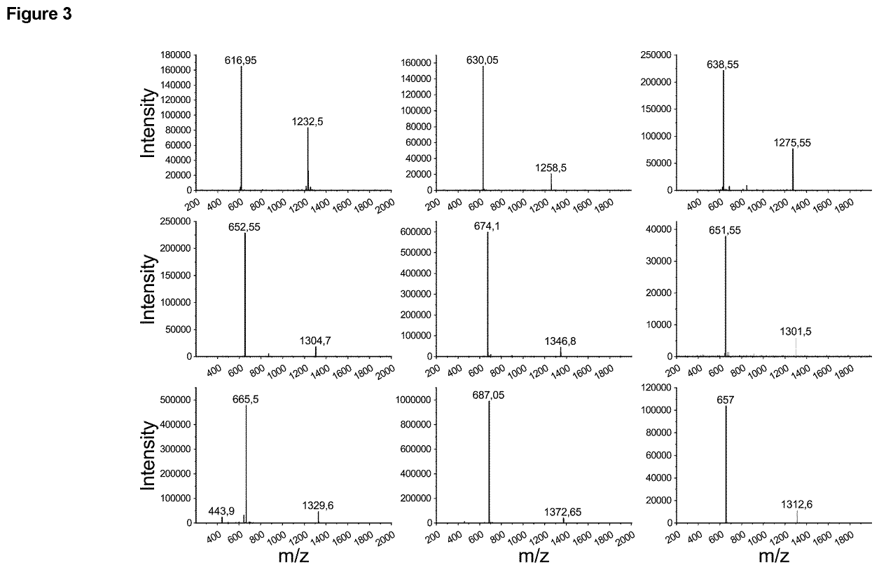

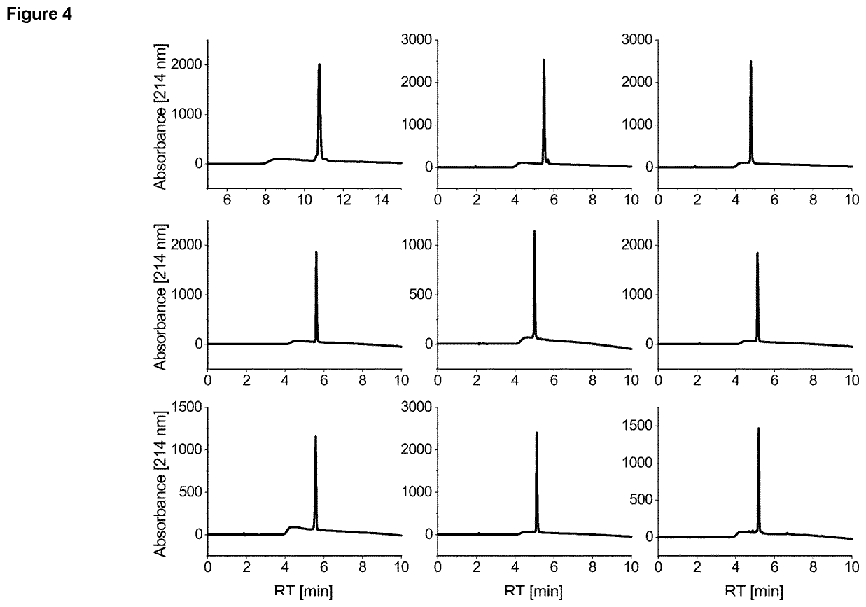

In detail, the resulting peptides A to I (cf. Table 1) were synthesized and characterized (FIG. 3, FIG. 4).

TABLE 1
Overview of Peptides.

| SEQ | | | |
|---|---|---|---|
| ID NO: | Abbreviation | Sequence | Origin |
| 1 | A | GAAGLLGG | PICS |
| 2 | B | GPAGLLGG | PICS |
| 3 | C | GAAGLRGG | PICS |
| 4 | D | GAAGLLGK | PICS |
| 5 | E | GAAGLRGK | PICS |
| 6 | F | GPAGLRGG | PICS |
| 7 | G | GPAGLLGK | PICS |
| 8 | H | GPAGLRGK | PICS |
| 9 | I | GPPGLTGP | Literature |
| | | | search |

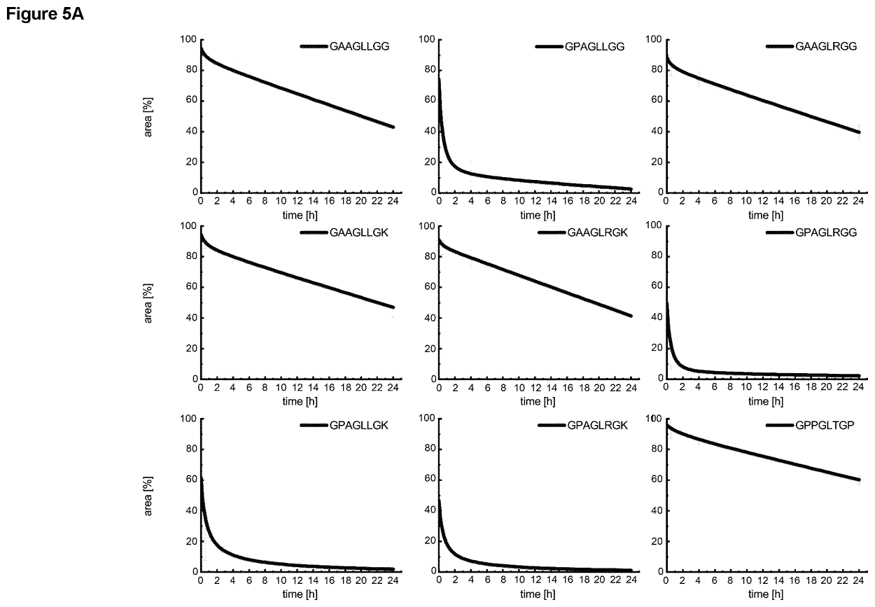

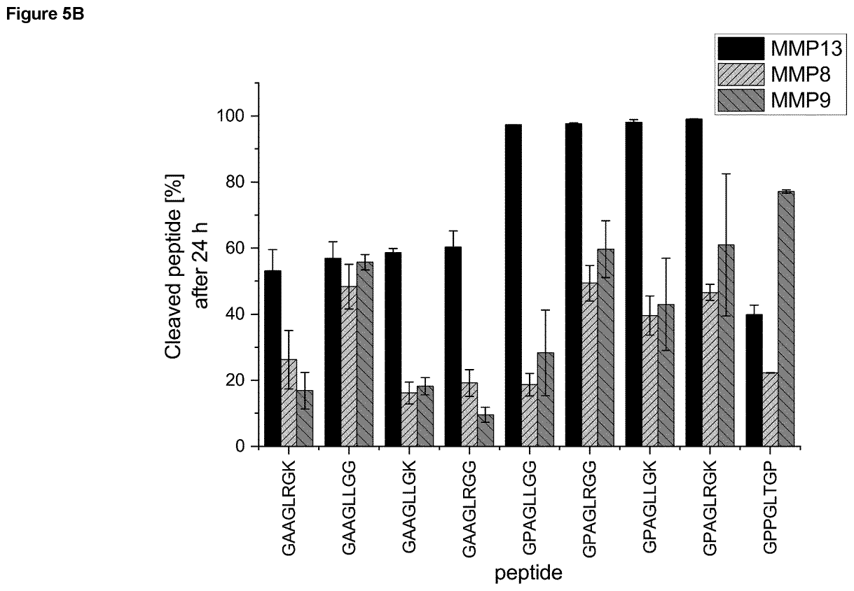

Subsequently, they were brought into contact with MMP-13 and were assayed as to their cleavage rate (FIG. 5).

This revealed that peptides having proline at position P3 were generally cleaved faster than those having alanine at P3. Yet, the latter was not true for the sequence of the natural substrate, which was cleaved the slowest.

### Example 3

In order to further evaluate specificity of these peptides of the invention, the cleavage rate by MMP-8 and MMP-9 was additionally examined (FIG. 5). All peptides were cleaved the most by MMP-13 in a time period of 24 h—except from the sequenced derived from the literature, for which cleavage by MMP-9 was stronger.

Among others, it was found that the sequence GPAGLLGG (SEQ ID NO: 2) is characterized by a high cleavage rate by MMP-13 and a low cleavage rate by MMP-8 and MMP-9, whereas the sequence GAAGLLGG (SEQ ID NO: 1) was cleaved equally strong by all three MMPs.

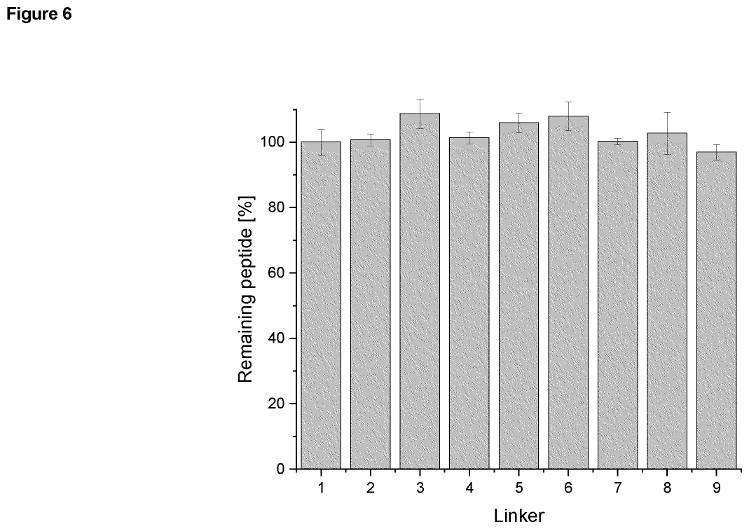

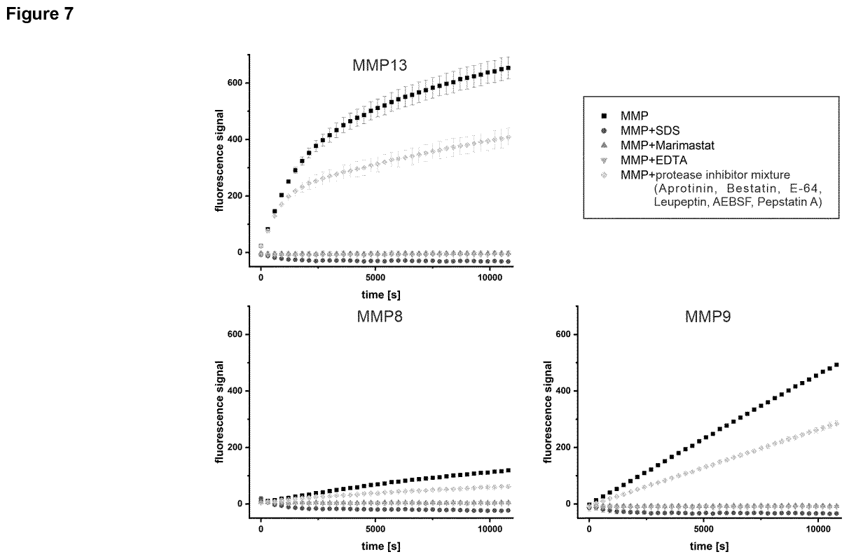

Furthermore, a stability assay with human plasma was performed (FIG. 6). All nine peptides were stable over a time period of 24 h at 37 °C and are, thus, suitable for systemic application in this respect.

### Example 4

A possible application of the peptides is the analysis of protease activity and the effect of substances on it. This can be utilized for understanding the role of proteases in physiological and pathological processes as well as for targeting proteases for disease diagnosis or treatment. As demonstration of such use, we synthesized a Forster resonance energy transfer (FRET) peptide with the sequence SGSG-K(Dnp)-GPAGLLGG-K(Mca)-GSGS (SEQ ID NO: 16). 7-Methoxycoumain-4-acetic acid (Mca) is a is a fluorescent dye with an excitation peak at 322 nm and an emission peak at 381 nm. The absorbance spectrum of 2,4-dinitrophenyl (Dnp) overlaps with the emission spectrum of Mca, therefore it acts as a quencher. As long as both are in close proximity (up to about 5-10 nm), excitation of Mca leads to energy transfer to Dnp. Separation of the fluorophore-quencher pair by hydrolysis of the protease-specific cleavage site between both compounds liberates the fluorescence signal and the rise in its intensity can easily be monitored overtime. This method is sensitive and time efficient. It allows quantitative assessment of enzymatic activity and the developed fluorogenic substrates are suitable for high throughput screening, for example of protease inhibitors.

Here, we used the FRET peptide to compare the inhibition of MMP13, MMP8 and MMP9 by EDTA, Marimastat, SDS and a commercial protease inhibitor mixture (Pierce™ Protease Inhibitor Tablets, EDTA-free, Catalog number A32965, containing Aprotinin, Bestatin, E-64, Leupeptin, AEBSF and Pepstatin A).

Incubation with MMP13 resulted in fast cleavage of the peptide and the highest fluorescence signal measurement. In 3 h, the activity of MMP13 was reduced to 62.5% by the protease inhibitor mixture. MMP9 cleaved the peptide more slowly and its activity was reduced to 57.1% by the protease inhibitor mixture. MMP8 showed the lowest cleavage rate for the substrate and its activity was reduced to 51.8% by the protease inhibitor mixture. In all cases, the addition of EDTA, Marimastat and SDS lead to a complete loss of MMP activity (FIG. 10).

### LIST OF REFERENCES

1. Poreba, M., Protease-activated prodrugs: strategies, challenges, and future directions. FEBS J, 2020, 287(10): 1936-1969.
2. Matthew F. et al. The Structure, Regulation, and Function of Human Matrix Metalloproteinase-13. Critical Reviews in Biochemistry and Molecular Biology, 2002, 37(3): 149-166.
3. Hernandez M. et al. Matrix metalloproteinase-13 is highly expressed in destructive periodontal disease activity. J Periodontol, 2006, 77(11): 1863-1870.
4. Özler K. et al. Serum and knee synovial fluid matrix metalloproteinase-13 and tumor necrosis factor-alpha levels in patients with late-stage osteoarthritis. Acta Orthop Traumatol Turc, 2016, 50(3): 356-361.
5. Moore B. A. et al. Induction of collagenase-3 (MMP-13) in rheumatoid arthritis synovial fibroblasts. Biochim Biophys Acta, 2000, 1502(2): 307-318.
6. Senthelal S. et al. Arthritis. StatPearls, 2022.
7. Hunter D. J. et al. Osteoarthritis in 2020 and beyond: a Lancet Commission. Lancet, 2020, 396(10264): 1711-1712.
8. Hootman J. M. et al. Updated Projected Prevalence of Self-Reported Doctor-Diagnosed Arthritis and Arthritis-Attributable Activity Limitation Among US Adults, 2015-2040. Arthritis Rheumatol, 2016, 68(7): 1582-1587.
9. Ma V. Y. et al. Incidence, prevalence, costs, and impact on disability of common conditions requiring rehabilitation in the United States: stroke, spinal cord injury, traumatic brain injury, multiple sclerosis, osteoarthritis, rheumatoid arthritis, limb loss, and back pain. Arch Phys Med Rehabil, 2014, 95(5): 986-995.
10. Raciborski F. et al. Diagnostic delays in rheumatic diseases with associated arthritis. Reumatologia, 2017, 55(4): 169-176.
11. Brophy R. H. et al. AAOS Clinical Practice Guideline Summary: Management of Osteoarthritis of the Knee (Nonarthroplasty), Third Edition. J Am Acad Orthop Surg., 2022, 30(9): e721-e729.
12. Fiehn C. et al. S2e-Leitlinie: Therapie der rheumatoiden Arthritis mit krankheitsmodifizierenden Medikamenten. Z Rheumatol, 2018, 77: 35-53.

1. A diagnostic sensor comprising:
a quenching moiety R1,
an MMP-13-sensitive peptide Pep, and
a signaling moiety R2,
wherein said quenching moiety R1 suppresses a signal generated by said signaling moiety R2 in said sensor.

2. The sensor of claim 1, wherein:
A) the MMP-13-sensitive peptide Pep comprises amino acid residues having the sequence P4-P3-P2-P1-P1′-P2′-P3′-P4′ (SEQ ID NO: 10), further wherein
P4 is G,
P3 is independently selected from A and P, preferably wherein P3 is P,
P2 is A,
P1 is G,
P1′ is L,
P2′ is independently selected from L and R,
P3′ is G, and
P4′ is independently selected from G and K;
Or,
B) the MMP-13-sensitive peptide Pep comprises amino acid residues having the sequence GPPGLTGP (SEQ ID NO: 9).

3. The sensor of claim 1, wherein:
i) the MMP-13-sensitive peptide Pep comprises amino acid residues having a sequence selected from the group consisting of GAAGLLGG (SEQ ID NO: 1), GAAGLRGG (SEQ ID NO: 3), GAAGLLGK (SEQ ID NO: 4), and GAAGLRGK (SEQ ID NO: 5); or,
ii) the MMP-13-sensitive peptide Pep comprises amino acid residues having a sequence selected from the group consisting of GPAGLLGG (SEQ ID NO: 2), GPAGLRGG (SEQ ID NO: 6), GPAGLLGK (SEQ ID NO: 7), and GPAGLRGK (SEQ ID NO: 8); or,
iii) the MMP-13-sensitive peptide Pep comprises amino acid residues having the sequence GPPGLTGP (SEQ ID NO: 9).

4. The sensor of claim 1, further comprising,
a first additional moiety L1,
a second additional moiety L2,
further wherein said sensor has a structure selected from the group consisting of R1-L1-Pep-L2-R2, L1-R1-Pep-L2-R2, L1-R1-Pep-R2-L2 and R1-L1-Pep-R2-L2.

5. The sensor of claim 1, wherein the quenching moiety R1 comprises a moiety selected from the group consisting of commercially available quenching moieties,
modified 4-((4-(Dimethylamino)phenyl)azo)benzoic acid, acetylated moieties, and Ac-K(Dabcyl).

6. The sensor of claim 1, wherein the fluorophore R2 comprises a moiety selected from the group consisting of small organic dyes, fluorescent proteins, and quantum dots.

7. The sensor of claim 1, wherein:
A) L1 comprises a moiety selected from the group consisting of small peptide linkers, natural polymers and synthetic polymers,
and/or
B) L2 comprises a moiety selected from the group consisting of small peptide linkers, natural polymers and synthetic polymers.

8. An MMP-13-sensitive peptide having a length of from 8 to 16 amino acids, wherein:
A) the MMP-13-sensitive peptide Pep comprises amino acid residues having the sequence P4-P3-P2-P1-P1′-P2′-P3′-P4′ (SEQ ID NO: 10), wherein
P4 is G,
P3 is independently selected from A and P,
P2 is A,
P1 is G,
P1′ is L,
P2′ is independently selected from L and R,
P3′ is G, and
P4′ is independently selected from G and K,
or
B) the MMP-13-sensitive peptide Pep comprises amino acid residues having the sequence GPPGLTGP (SEQ ID NO: 9).

9. The peptide of claim 8, wherein the peptide comprises:
i) amino acid residues having a sequence selected from the group consisting of GAAGLLGG (SEQ ID NO: 1), GAAGLRGG (SEQ ID NO: 3), GAAGLLGK (SEQ ID NO: 4), and GAAGLRGK (SEQ ID NO: 5); or
ii) amino acid residues having a sequence selected from the group consisting of GPAGLLGG (SEQ ID NO: 2), GPAGLRGG (SEQ ID NO: 6), GPAGLLGK (SEQ ID NO: 7), and GPAGLRGK (SEQ ID NO: 8); or
iii) amino acid residues having the sequence GPPGLTGP (SEQ ID NO: 9).

10. A diagnostic kit comprising the sensor of claim 1.

11. The sensor of claim 1, constructed for use in medicine.

12. A method of diagnosing cancer or periodontitis in a human subject,
said method comprising the step of using the sensor of claim 1 to determine MMP-13 activity in a subject,
wherein an increased level of MMP-13 activity relative to a control level is indicative of the presence of cancer or periodontitis in said subject.

13. The method of claim 12, wherein said method comprises the steps of:
a) determining a level of MMP-13 activity in the subject using the sensor of claim 1,
b) comparing the level of MMP-13 activity to a control level of MMP-13 activity,
c) diagnosing the subject with cancer or periodontitis when the level of MMP-13 activity determined in step a) is greater than the control level recited in step b).

14. A method of determining MMP-13 activity in a sample, wherein the method comprises using the kit in accordance with claim 10;
further wherein
A) the sample is obtained from the oral cavity of a subject,
and
B) an increased level of MMP-13 activity relative to a level of a control sample is indicative of the subject having cancer or periodontitis.

15. The method of claim 14, wherein the method comprises the steps of:
a) determining a level of MMP-13 activity in a sample obtained from the subject,
b) comparing the level in the said sample to a control level of MMP-13 activity,
c) diagnosing the subject with cancer or periodontitis when an increased level of MMP-13 activity relative to the level of said control is indicative of the subject having cancer or periodontitis.

16. A method of using the peptide of claim 8 in preventing or treating a disease in a human subject,
wherein the disease is selected from the group consisting of cancer, oral inflammation, periodontitis, degenerative arthropathies and inflammatory arthropathies.

17. The method of claim 16, wherein said peptide is part of a drug conjugate comprising said peptide in combination with an active agent, wherein the active agent is released from the drug conjugate upon cleavage with MMP-13.

18. The sensor of claim 2, wherein the MMP-13-sensitive peptide Pep has the sequence P4-P3-P2-P1-P1′-P2′-P3′-P4′ (SEQ ID NO: 10), wherein P4′ is K.

19. The sensor of claim 6, wherein said fluorophore R2 is selected from the group consisting of 5-((2-Aminoethyl)amino)naphthalene-1-sulfonic acid and E(Edans).

20. A diagnostic kit comprising the kit of claim 8.
